(19) United States
(12) Patent Application Publication
(10) Pub. No.: US 2023/0285044 A1
(43) Pub. Date: Sep. 14, 2023

(54) LAPAROSCOPIC SURGICAL INSTRUMENT

(71) Applicant: Alma Mater Studiorum - Universita 'di Bologna, Bologna (IT)

(72) Inventors: Vincenzo PARENTI CASTELLI, Bologna (IT); Fabio MANFERRARI, Bologna (IT); Nicola SANCISI, San Lazzaro Di Savena (IT); Rocco VERTECHY, Pietrasanta (IT); Giulia AVALLONE, Bologna (IT); Enrico FEDERICI, Bologna (IT); Filippo BATTOCCHI, San Secundo (IT); Leonardo MAGNANI, Gabicee Mare (IT); Luca LUZI, Dozza (IT)

(73) Assignee: Alma Mater Studiorum - Universita 'di Bologna, Bologna (IT)

(21) Appl. No.: 18/040,802

(22) PCT Filed: Aug. 7, 2020
(86) PCT No.: PCT/IT2020/000060

§ 371 Date: Feb. 6, 2023

(51) Int. Cl. A61B 17/32; A61B 17/3201

(52) U.S. Cl. CPC A61B 17/320016; A61B 17/3201; A61B 2017/2929

(57) ABSTRACT

A laparoscopic surgical instrument including a connection assembly, an end effector, and a manipulation unit. The instrument also includes a means for transmitting motion from the manipulation unit to the end effector. The means for transmitting motion may include a means for transmitting first rotational movement of the manipulation unit to the end effector in a way such that the end effector rotates around an axis that is orthogonal to the axis of the connection assembly. The means for transmitting motion may also include a means for transmitting a second rotational movement to the end effector in a way such that the end effector rotates around it's own axis that is parallel to the axis of the manipulation unit.

### TECHNICAL FIELD OF INVENTION

The present invention relates to a laparoscopic surgical instrument. More particularly, the invention deals with a hand-held instrument equipped with a highly dexterous extremity, making the execution of very precise surgical intervention as in the robotic system while keeping very low costs as for all the hand-held surgical instrument. The present invention relates also to a kit comprising said laparoscopic surgical instrument and an instrument holder (e.g. a trocar) for the insertion of said instrument through an incision into a patient cavity.

### BACKGROUND

Laparoscopic surgery, also known as Minimally Invasive Surgery (MIS) is a surgical technique developed in the late '50. Thanks to laparoscopy, surgical technique surgeons can manipulate the internal tissues of the patients remaining with their hands away from the surgical field. The surgical instruments are inserted inside the body of the patient via small skin incisions and the tool at the extremity of each instrument is controlled by the surgeon. More particularly, the laparoscopic instruments are introduced into the patient's cavity by means of an instrument holder, known as trocar. A trocar is a surgical device that is made up of an obturator and a cannula (i.e. a hollow tube), and a seal. Trocars are placed through the abdomen during laparoscopic surgery and are used as portal for the subsequent placement of other instruments, such as graspers, scissors, staplers, etc. The main advantages that the laparoscopic technique provides to the patients are the reduction of the invasiveness of the surgery (i.e. less pain and less risks of haemorrhages and infections) due to smaller incisions and a faster recovery times after surgery, consequently causing a reduction of social health system costs.

As known, surgical instruments have to fulfil a number of requirements such as small size, efficiency in terms of degrees of freedom the surgeon can exploit, reliability/robustness of the instrument and, finally, costs which should be contained. Surgeons can control the tool at the extremity of the instrument: (i) remotely, i.e. by using a robotic system or (ii) directly, i.e. by using hand-held surgical instruments; and laparoscopic surgical instruments can be classified on the basis of the mobility given to the tool at the extremity of the instrument. In this context, it has to remember that, a trocar (and therefore the instrument placed in it) has four degrees of freedom: one translational along the axis of the cannula and three rotational. The three allowable rotations are around the axis of the cannula and around the two axes perpendicular to the cannula. Each laparoscopic instrument that can be inserted in a trocar, has, thus, the above mentioned four degrees of freedom, that are also known in the field as “gratuitous” degrees of freedom.

That said, the hand-held instruments have only the four “gratuitous” degrees of freedom and usually have a fixed extremity, or end effector, and, at most, surgeons can control only the opening/closing of the tool. Robotic systems, instead, usually, employ surgical instruments with dexterous extremity, or end-effector, and i.e. surgeons can control also movements of the tool other than the “gratuitous” degrees of freedom.

The main advantages of hand-held surgical tools are that they are less expensive than the instruments used in robotic systems and usually requires a lower surgery time, obviously for trained surgeons. On the other hand, the advantages of the robotic systems are precision and repeatability in the execution of surgical tasks, tool stability, force tool-tissue control and surgeon-hand-tremor reduction, whereas the main drawbacks are high purchase and maintenance costs of the robotic system, higher encumbrances and high pre-surgery time to set up the robot for the patient. Within this context, there is a need to design hand-held surgical instruments that can provide a good compromise between high efficiency and low surgery time and costs. In other words, there is a need of and hand-held instruments that allows for high precision and control in the execution of the surgeon's movement, comparable with those of the instruments employed in the robotic systems.

Moreover, the robotic tools, known in the prior art, in addition to the disadvantages consisting in the high costs involved, have not few limitations relatively to the allowable movements to the end effector. The majority of the surgical robotic tools are provided with a central and rectilinear body that transmit to the end effector the movements imparted via console of the robotic system. A part from the movements of opening and closing of the end effector (when it is for example a clamp) and the four “gratuitous” movements of the trocar holding the instrument, the allowable movements, usually, consists only in a rotational movement around an axis that is orthogonal to the central body. Among the most advanced robotic tools, there are also someone wherein two rotational movements of the end effector are allowable, other than the opening/closing of the end effector and, than the four “gratuitous” degrees of freedom of the instrument as a whole. The first one of such movements is the rotation cited above around a first axis and the second one is a rotation around a second axis orthogonal to the first axis mentioned above and orthogonal to the central body.

However, even in such advanced robotic tools, the motion of the end effector around its own axis is not permitted, and the dexterity is limited by the fact that the first axis, the second axis are not incident at an only one point. The second axis and the first axis are skew lines and, thus, the two provided rotations do not occur around a fixed center. Moreover, in such instruments, it lacks the possibility to perform a rotation of the end effector around the own axis, when the latter is not aligned with the rectilinear central body.

In other words, at best of our knowledge, no one of the known robotic instruments, or more in general, of the known dexterous extremity instruments, is able to provide a motion of the end effector that is assimilable to the one provided by a spherical joint positioned in correspondence of the point of intersection between the central body and the end effector. This last condition is the one that allows for the wider range of possible maneuvers executable by the surgeons and the best control of the instrument. Hence, in the state of art, there is also a need to improve the dexterity of the surgical instruments of the robotic system.

In addition to the state of art limitations described above, in the majority of the known instruments (robotic and hand-held) only the closing of end effector can be controlled by the surgeon, whereas the opening of the same occurs by means of a spring without any possibility of control. In some instrument, mechanisms are provided to guarantee the control of the opening operations of the end effector, but such mechanisms are such complex that requires an increasing of the dimensions of the end effector, or more precisely an increasing of the space necessary for the connection between the arms of the end effector and the central body of the instrument. For these reasons, there is also a need in the current state of art, for mechanisms for opening and closing of the end effector that allows for a surgeon's fine control of both the operations and that, at the same time, is simple and of contained dimension so to maintain not excessive long the distance between the central body and the arms of the end effector.

Finally, an important issue currently unsolved in the prior art laparoscopic instrument, deals with the force exerted by the crossed tissues during surgical suturing operations. During execution of a surgical suture, indeed, the surgeon has to perform the movements around the first axis cited above and, when the tissues are crossed by the suturing needle held by the end effector, a torque can be generated by the same tissues on the end effector and, thus, on the central body. This torque can cause an unintentional rotation of the end effector, when the instrument is in working bended configurations, and, thus, of the central body, around their own axes. Therefore, in the available instrument nowadays, there is a need of finding a way to compensate the unintentional rotation generated by the tissues on which the surgeons has to intervene.

### SUMMARY OF THE INVENTION

A first object of the present invention is, therefore, to provide an instrument that conjugates the advantages of the hand-held instrument of the prior art relatively in terms of low surgery time and low costs and the advantages of the robotic tools in terms of precision and control during the execution of the surgical interventions.

A second object of the present invention is to provide a hand-held instrument that enlarges the range of possible maneuvers executable by the surgeons with respect to those allowable by the instruments of robotic systems.

In particular, the present invention provides an instrument where at least two rotational movements of the end effector are allowed, said at least two rotational movements being around two incident axes. More particularly, the first rotational movement is around an axis belonging to a plane orthogonal and incident to the axis of the central body at the point in which the end effector is connected to the central body and the second rotational unit is around the end effector's own axis. Since the central body has a prismatic or cylindrical shape, the axis of the central body can be also referred to with the wording “longitudinal axis”.

In order to perform said rotational movements the instrument of the present invention comprises:
a connection assembly having an axis (or a longitudinal axis), a first end and a second end;
an end having an its own axis of rotation incident to the longitudinal axis at the second end, said end effector being mounted on the second end of the connection assembly;
a manipulation unit having an its own axis of rotation incident to the longitudinal axis at the first end, said manipulation unit being mounted on the first end of the connection assembly;
means for transmitting, to the end effector, a first rotational movement of the manipulation unit around a first axis belonging to a plane orthogonal to the longitudinal axis and incident to the longitudinal axis at the first end of the connection assembly, in such a way that the end effector rotates around an axis orthogonal to the longitudinal axis and incident to the longitudinal axis at the second end of the connection assembly; and
means for transmitting, to the end effector, a second rotational movement of the manipulation unit around its own axis, in such a way that the end effector rotates around its own axis, the axis of the end effector being parallel to the axis of the manipulation unit.

The means for transmitting the second rotational movement comprises, substantially, two gear assembly, each gear assembly being formed by two bevel gears with coinciding vertexes, connected by a tube housed in a tubular frame of the connection assembly. The first gears is placed between the manipulation unit and the tube in the frame and is configured for transmitting the first rotational movement from the manipulation unit to the shaft; and the second gear is placed between the tube and the end effector, and is configured for transmitting the first rotational movement from the shaft to the end effector.

The means for transmitting the second rotational movement comprises, substantially, two Cardan joints connected by a shaft housed in the tube connecting the two gears assembly. The first Cardan joint is placed between the manipulation unit and the shaft in the frame in such a way that its center coincides with the vertexes of the bevel gears of the first gear assembly, ad is configured for transmitting the second rotational movement from the manipulation unit to the shaft. The second Cardan joint is placed between the shaft and the end effector in such a way that its center coincides with the vertexes of the bevel gear of the second gear assembly, and is configured for transmitting the second rotational movement from the shaft to the end effector.

In addition to the two rotational movements cited above, the instrument of the present invention can be used to transmit another, or third, rotational movement from the manipulation unit to the end effector, said another, or third, rotational movement being around an axis, orthogonal to the longitudinal axis and the first axis and different from the end effector's own axis of rotation.

The third rotational movement can be performed by the instrument of the present invention if, starting from a position in which the manipulation's unit own axis, and the end effector's own axis are both aligned with the longitudinal axis of the connection assembly, the manipulation unit is rotated of 90° around its own axis and, then, rotated around to the second axis. The rotation of the manipulation unit around the second axis can be, thus, transmitted to the end effector, thanks to the same means for transmitting the first rotational movement from the manipulation unit to the end effector.

Since the second axis is incident to the first axis and the end effector's own axis at the point wherein the end effector is connected to the central body, the three performable rotational movement are assimilable to three rotations of a spherical joint positioned in correspondence of the point of intersection between the central body and the end effector. Finally, the dexterity of the end effector, is further improved by providing means for rotating the end effector, independently from the manipulation unit. Said means comprise:
a drum brake comprising a drum pulley firmly connected with a manipulation unit's shaft firmly connected with the manipulation unit and connected to the shaft placed in the frame be means of the first Cardan joint; and
a handling ring firmly connected with the drum pulley in such a way that a rotation of the handling ring around its own axis causes a rotation of the drum pulley and, thus, of the manipulation unit's shaft and, consequently, of the shaft placed in the frame around its own axis.

A third object of the present invention is, then, to provide a hand-held instrument comprising a mechanism for opening and closing of the end effector that allows for a surgeon's fine control of both the operations and that, at the same time, is of reduced dimension so that the distance between the central body and the arms of the end effector is not too long. To this aim the instrument of the present invention comprise:
a connection assembly having an axis;
an end effector mounted on a first end of the connection assembly, said end effector comprising:
   a first arm;
   a second arm;
   a pulley firmly connected with the first arm;
   a first gear firmly connected with the second arm;
   a second gear forming a gear assembly with the first gear, said second gear being concentric and integrally connected with the pulley; and
a manipulation unit mounted on a second end of the connection assembly, said manipulation unit comprising:
   a trigger;
   a pulley firmly connected with the trigger; and
   an idle pulley.

In order to allow for the movement of the two arms, determining the opening and closing of the end effector the instrument comprises means for transmitting motion from the manipulation unit to the end effector, comprising a cable, not shown in the figures, which connects the pulley of the manipulation unit with the pulley of the end effector, said cable being divided into two portions by means of the idle pulley, before to be wrapped around the pulley of the manipulation unit.

Finally, a fourth object of the present invention is to provide an instrument that is able to compensate the unintentional rotation generated by the tissues on which the surgeons has to intervene, with particular reference to the torque generated by the tissues crossed during the operations of surgical suture. To this aim the present invention comprises: a blocking device mounted on the frame, said device comprising:
a support structure with means for docking the device to an instrument holder for at least a partial insertion of said laparoscopic surgical instrument through an incision into a patient cavity;
two truncated cone rolls (for the purposes of this description, the term “truncated cone roll” means a curvilinear generator revolution solid), the frame being placed between the two truncated cone rolls; and
a lever configured to allow the two truncated cone rolls to translate from a first position in which the two truncated cone rolls are not in contact with the frame to a second position in which the two truncated cone rolls are in contact with the frame preventing by friction the rotation of the frame around the longitudinal axis, the translation of the frame along the longitudinal axis being maintained allowable.

For the same object, it forms subject of the present invention also a kit comprising:
the laparoscopic surgical instrument comprising the blocking element;
an instrument provided with means for docking the instrument holder to the blocking device, the means for docking the instrument holder being configured to cooperate with the means for docking of the support structure of the blocking device.

This and further objects of the present invention will be made clearer by the reading of the following detailed description of some preferred embodiments of the present invention, to be understood as a non-limiting example of the more general concepts claimed.

### BRIEF DESCRIPTION OF THE DRAWINGS

The description refers to the accompanying drawings, in which:

FIG. 1a is a schematic representation of the degree of freedom of a trocar of the prior art and any surgical laparoscopic instrument of the prior art inserted in such a trocar;

FIG. 2 is a schematic representation of the laparoscopic surgical instrument of the present invention showing the rotational movements transmitted from the manipulation unit to the end effector;

FIG. 3 is a side view of a fifth embodiment of the instrument of the present invention with the first shaft, the second shaft and the third shaft aligned and the closed clamp;

FIG. 4 is a side view of a fifth embodiment of the instrument of the present invention with the first shaft, the second shaft and the third shaft aligned and the open clamp;

FIG. 5a is a top view of a of a fifth embodiment of the instrument of the present invention with the first shaft and second shaft rotated around the first axis (U₁) and the axis (U₁′), respectively;

FIG. 5b is a side view of a fifth embodiment of the instrument of the present invention with the first shaft and second shaft rotated around the first axis (U₁) and the axis (U₁′), respectively;

FIG. 6a is a side view of a detail of the fifth embodiment of the present invention, said detail being relative to the means for transmitting the first rotational movement, in correspondence of the manipulation unit;

FIG. 6b is a side view of a detail of the fifth embodiment of the instrument the present invention, said detail being relative to the means for transmitting the first rotational movement, in correspondence of the end effector;

FIG. 7a is a top view of a fifth embodiment of the instrument of the present invention with the first shaft and second shaft rotated around the second axis (U₂) and the axis (U₂′), respectively;

FIG. 7b is a side view of a fifth embodiment of the instrument of the present invention with the first shaft and second shaft rotated around the second axis (U₂) and the axis (U₂′), respectively;

FIG. 8a is a side view of a detail of the fifth embodiment of the instrument of the present invention, said detail being relative to the means for transmitting the second rotational movement, in correspondence of the manipulation unit;

FIG. 8b is a side view of a detail of the fifth embodiment of the instrument of the present invention, said detail being relative of the means for transmitting the second rotational movement, in correspondence of the end effector;

FIG. 9a is a side view of a detail of the fifth embodiment of the instrument the present invention, said detail being relative to the brake block;

FIG. 9b is a top view of a detail of the fifth embodiment of the instrument the present invention, said detail being relative to the brake block;

FIG. 10a is a perspective view of the fifth embodiment of the instrument of the present invention the first shaft, the second shaft and the third shaft being aligned;

FIG. 10b is a perspective view of the fifth embodiment of the instrument of the present invention, when the first shaft is rotated, around the second axis (U₂), of an angle less than 90° with respect to the third shaft, the second axis (U₃) being coincident with the axis (U₃) of the connection assembly and;

FIG. 10c is a perspective view of the of the fifth embodiment of the instrument of the present invention, when the first shaft is rotated, around the second axis (U₂), of an angle of ° 90°, with respect to the third shaft, the second axis (U₂) being coincident with the axis (U₃) of the connection assembly;

FIG. 11 is a perspective view of the of the fifth embodiment of the instrument of the present invention when the first shaft is rotated, around the second axis (U₂), of an angle of 90°, with respect to the third shaft and, around a fourth axis (U₄) orthogonal to the axis (U₃) and to the first axis (U₁), the second axis (U₂) being coincident with the axis (U₃) of the connection assembly;

FIG. 12 is a schematic representation of a sixth and thirteenth embodiments of the laparoscopic surgical instrument of the present invention;

FIG. 13 is a schematic representation of a detail of the sixth and thirteenth embodiment of the laparoscopic surgical instrument of the present invention, said detail being relative to an example of flexure hinge comprised in the means for transmitting the first rotational movement;

FIG. 14 is a schematic representation of a sixth and thirteenth embodiment of the laparoscopic surgical instrument of the present invention;

FIG. 15 is a longitudinal section of a detail of the instrument of the present invention, said detail being relative to the manipulation unit;

FIG. 16a is a first perspective view of the detail of the fifth embodiment of the instrument of the present invention, said detail being relative to the end effector;

FIG. 16b is a second perspective view of the detail of the end effector of the fifth embodiment of the instrument of the present invention said detail being relative to the end effector;

FIG. 17 is a perspective view of a detail of the instrument of the present invention, said detail being relative to block element of the spontaneous rotation of the axis of the connection assembly of the present invention;

FIG. 18a is a perspective view of detail of the kit of the present invention, said detail being relative to block element of the spontaneous rotation axis of the connection assembly of the present invention;

FIG. 18b is a perspective view of detail of the kit of the present invention, said detail being relative to the trocar;

FIG. 19a is a side view of a detail of the instrument of the present invention, said detail being relative to the first embodiment of the handle; and

FIG. 19b is a side view of a detail of the instrument of the present invention, said detail being relative to a second embodiment of the handle.

### DETAILED DESCRIPTION OF THE PREFERRED EMBODIMENTS

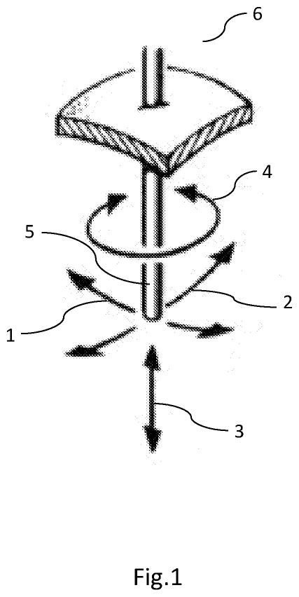

Referring to FIG. 1, and as already mentioned above, the instruments of the prior art when in use, are inserted in an instrument holder, known as trocar (6), that guides the insertion of the laparoscopic surgical instrument through an incision into a patient cavity. The trocar (6), and, therefore, whichever instrument inserted in it, has four degrees of freedom. The first one is a translational degree of freedom (3) along the axis of the rectilinear cannula (5) of the trocar (6). The other three degrees of freedom are rotational degrees. They are, in particular: a first rotational degree (4) around an axis parallel to the cannula (5); a second rotational degree (1) around a first axis perpendicular to the cannula (5); and a third rotational degree (2) around a second axis, said second axis being perpendicular to the first axis.

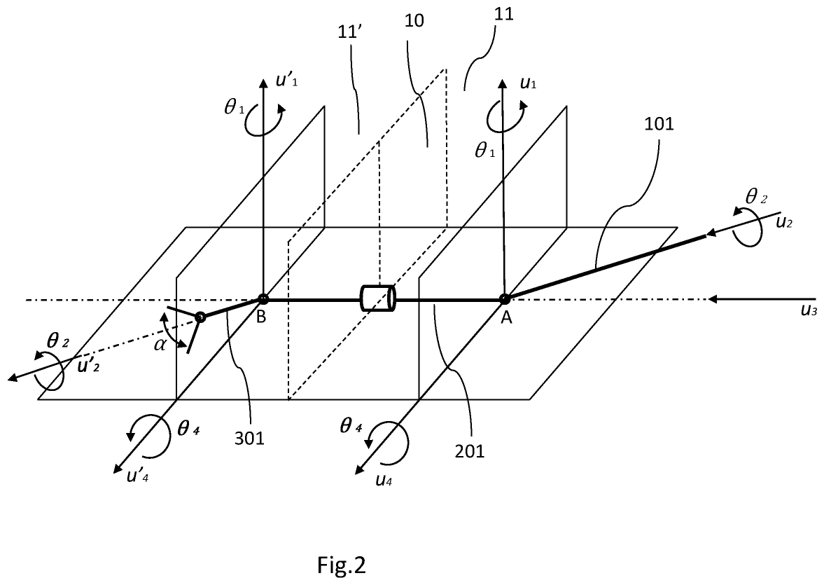

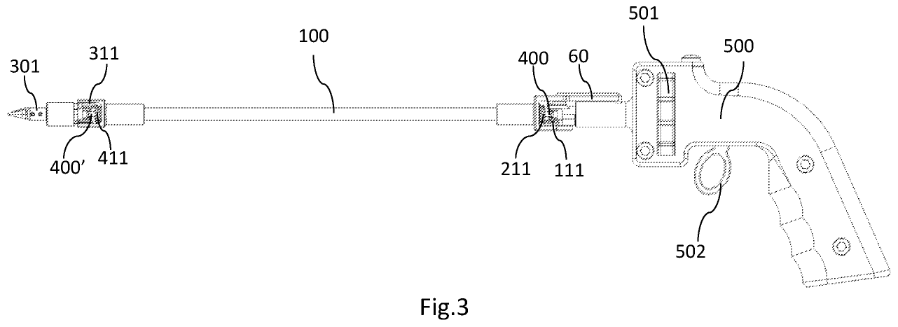

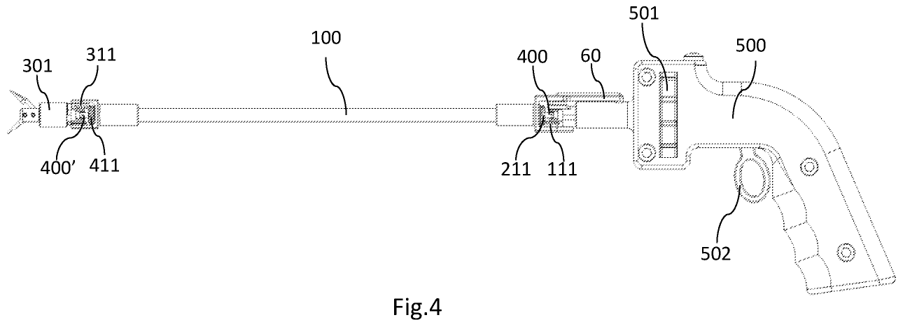

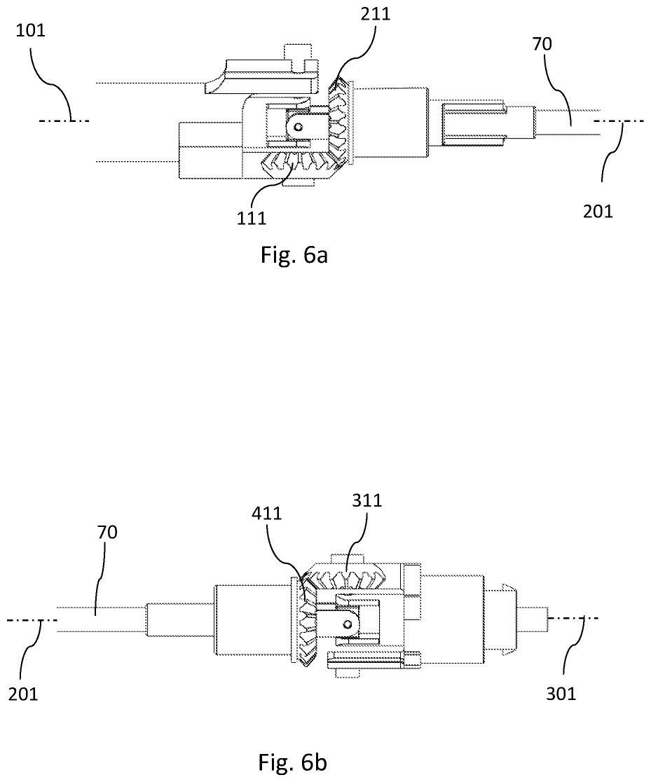

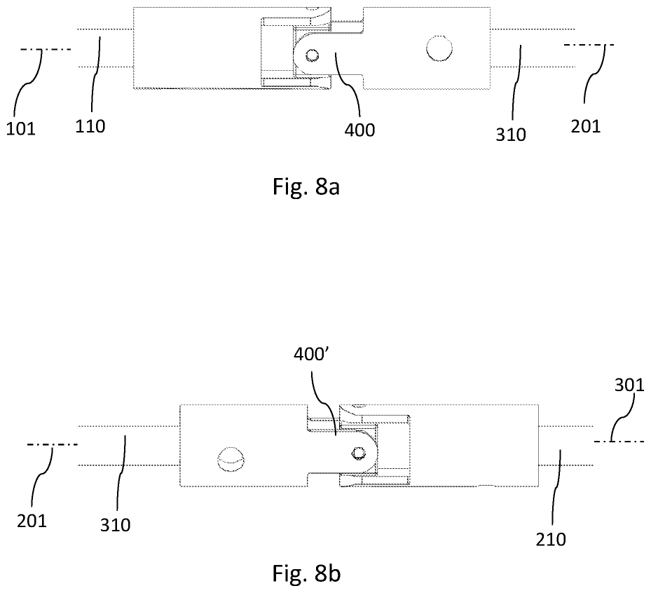

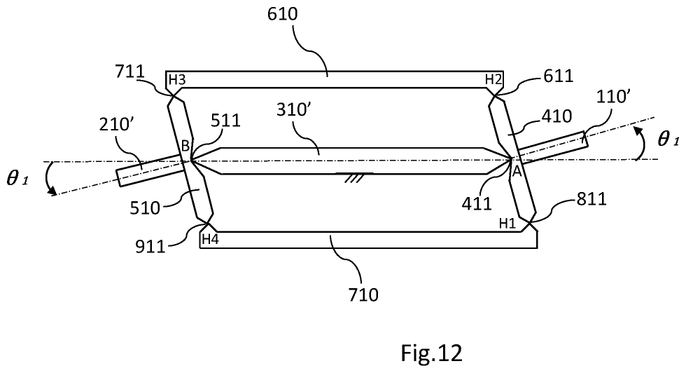

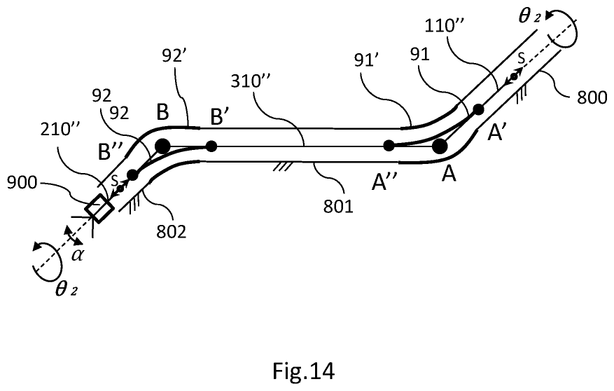

Referring to FIGS. 2, 3, 4, 6a, 6b, 8a, 8b, 12 and 14, the instrument of the present invention comprises:
a connection assembly (201) having an axis (U₃), a first end (A) and a second end (B);
an end effector (301) having an its own axis (U₂) of rotation incident to the axis (U₃) of the connection assembly (201) at the second end (B), said end effector (301) being mounted on the second end (B) of the connection assembly (201);
a manipulation unit (101) having an its own axis (U₂) of rotation incident to the axis (U₃) of the connection assembly (201) at the first end (A), said manipulation unit (101) being mounted on the first end (A) of the connection assembly (201);
means for transmitting motion (111, 211, 311, 411, 110, 210, 310, 400, 400′, 310′, 110′, 210′, 410, 510, 610, 710, 411, 511, 611, 711, 811, 911, 110″, 210″, 310″, 91, 92) from the manipulation unit (101) to the end effector (301);

The means for transmitting motion (111, 211, 311, 411, 110, 210, 310, 400, 400′, 310′, 110′, 210′, 410, 510, 610, 710, 411, 511, 611, 711, 811, 911, 110″, 210″, 310″, 91, 92) comprise at least:
means for transmitting (111, 211, 311, 411, 310′, 110′, 210′, 410, 510, 610, 710, 411, 511, 611, 711, 811, 911) a first rotational movement of the manipulation unit (101) around a first axis (U₁) belonging to a plane orthogonal to the axis (U₃) of the connection assembly (201) and incident to the axis (U₃) of the connection assembly (201) at the first end (A) of the connection assembly (201), to the end effector (301), in such a way that the end effector (301) rotates around an axis (U₁′) orthogonal to the axis (U₃) of the connection assembly (201) and incident to the axis (U₃) of the connection assembly (201) at the second end (B) of the connection assembly (201); and
means for transmitting (110, 210, 310, 400, 400′, 110″, 210″, 310″, 91, 92) a second rotational movement of the manipulation unit (101) around its own axis (U₂) to the end effector (301), in such a way that the end effector (301) rotates around its own axis (U₂′), the axis (U₂′), of the end effector (301) being parallel to the axis (U₂) of the manipulation unit (101).

The instrument of the present invention, has, therefore, at least two rotational degrees of freedom (θ₁, θ₂), in addition to the degrees of freedom guaranteed by the movements of the trocar wherein the instrument have to be inserted when in use during the surgical interventions. The first rotational degree of freedom (θ₁) is associated to the first rotational movement described above and the second rotational degree of freedom (θ₂) is associated to the second rotational movement. Assuming that the instrument is inserted in the patient's cavity through a patient's surface (10), the working field of the instrument is divided into two main volumes: the first one (11) outside the patient's cavity and the second one inside the patient's cavity (11′). The rotations (θ₁, θ₂) of manipulation unit (101) are applied in the first volume (11) and are transmitted through means, coinciding with or placed in, the connection assembly (201), to the end effector, which rotates into the second volume (11′). The connection assembly (201), thus, passes through the patient's surface (10).

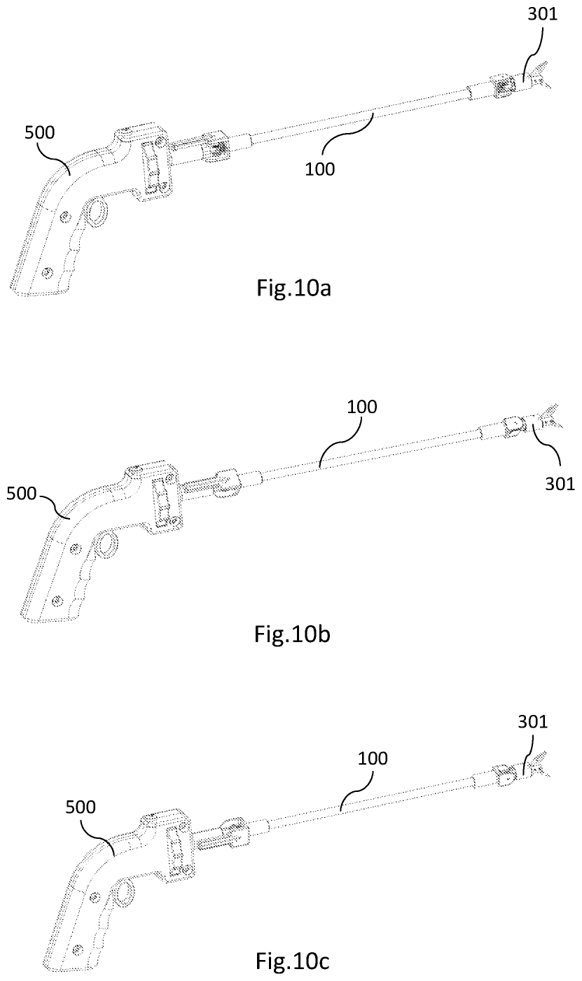

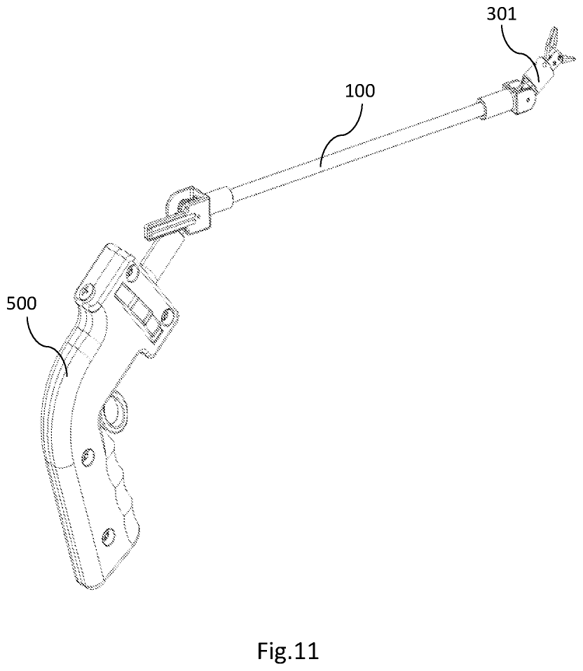

Referring to FIGS. 1, 2, 10a, 10b, 10c and 11, in addition to the two rotational degrees of freedom (θ₁, θ₂) cited above, the instrument of the present invention can be used to transmit another rotation (θ₄) from the manipulation unit (101) to the end effector (301), said another rotation (θ₄) being around a second axis (U₄), orthogonal to axis (U₃) of the connection assembly (201) and incident to the axis (U₃) of the connection assembly (201) at the first end (A) of the connection assembly (201). This another rotation (θ₄) is transmitted to the end effector (301) in such a way that the end effector (301) rotates around an axis (U₄) parallel to the second axis (U₄) and incident to the axis (U₃) of the connection assembly (201) at the second end (B) of the connection assembly (201).

Such additional rotation (θ₄) can be performed using the instrument as in the followings. Starting from a position in which the manipulation's unit (101) own axis (U₂), and the end effector's (301) own axis (U₂) are both aligned with the axis (U₃) of the connection assembly (201), the manipulation unit (101) can be rotated of 90° around its own axis (U₂) and then rotated around to the second axis (U₄), by using the same means that allow for the rotation of the manipulation unit (101) around the first axis (U₁). The rotation of the manipulation unit (101) around the second axis (U₄) can be, thus, transmitted to the end effector (301), thanks to the same means for transmitting the first rotational movement from the manipulation unit (101) to the end effector (301).

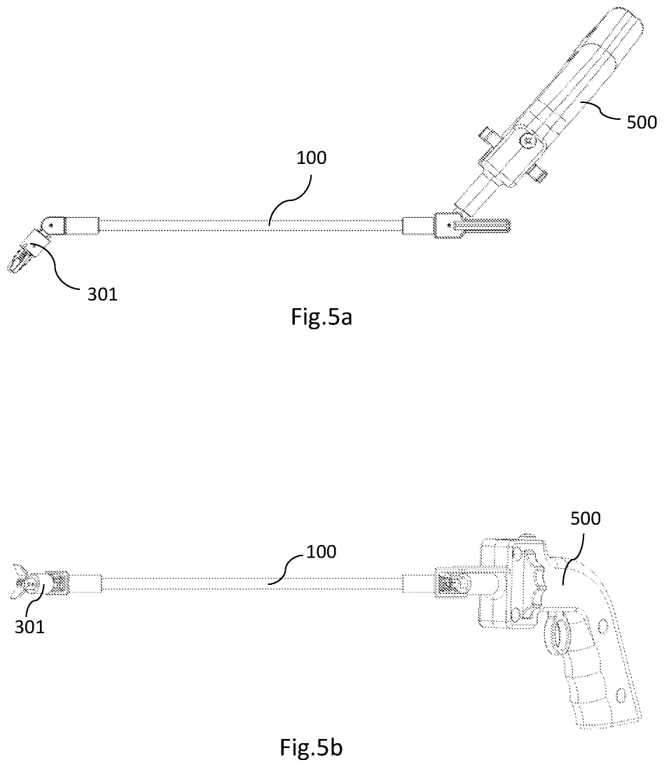

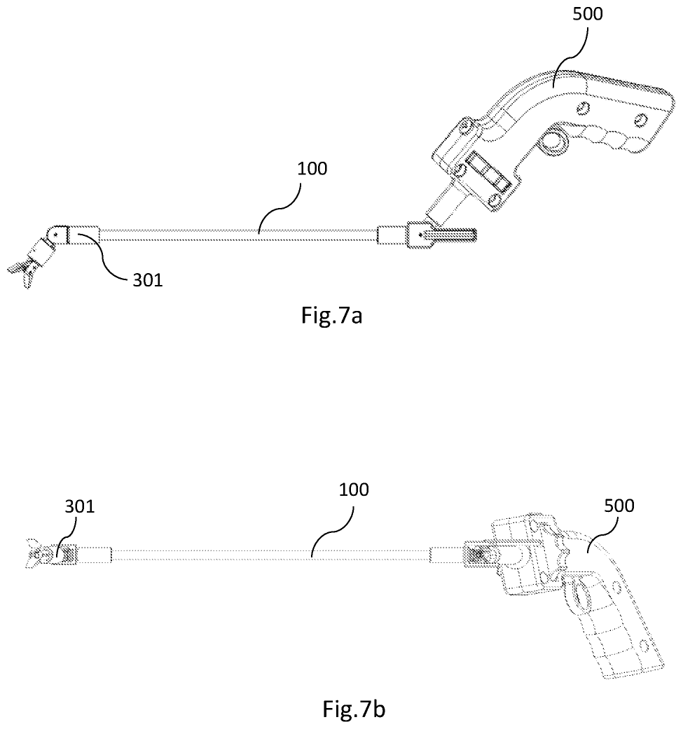

Referring to the FIGS. 2, 3, 4, 5a, 5b, 6a, 6b, 7a, 7b, 8a and 8b, a first embodiment of the instrument of the present invention, the connection assembly (201) comprises a cylindrical frame (100). The means for transmitting (111, 211, 311, 411) the first rotational movement comprise:
a first bevel gear (111) firmly connected with the manipulation unit (101) and having the vertex located in correspondence with the first end (A) of the connection assembly (201);
a second bevel gear (211) forming with the first bevel gear (111) a first gear assembly, in such a way that a rotation of the first bevel gear (111) around its own axis causes a rotation of the second bevel gear (211) around its own axis;
a third bevel gear (311) firmly connected with the end effector (301) and having the vertex in correspondence with the second end (B) of the connection assembly (201);
a fourth bevel gear (411) forming with the third bevel gear (311) a second gear assembly, in such a way that a rotation of the fourth bevel gear (411) around its own axis causes a rotation of the third bevel gear (311) around its own axis; and
a hollow tube (70) aligned with the axis (U₃) of the connection assembly (201) and hosed in the frame (100), said tube (70) cooperating with the second bevel gear (211) and the fourth bevel gear (411) in such a way that a rotation of the second bevel gear (211) around its own axis causes a rotation of the fourth bevel gear (411) around its own axis.

The means for transmitting (110, 210, 310, 400, 400′) the second rotational movement comprise:
a first shaft (110) firmly connected with the manipulation unit (101);
a second shaft (210) firmly connected with the end effector (301);
a third shaft (310) aligned with the axis (U₃) of the connection assembly (201), said third shaft (310) being housed in the tube (70);
a first Cardan joint (400) centered in correspondence of the first end (A) of the connection assembly (201); and
a second Cardan joint (400′) centered in correspondence of the second end (B) of the connection assembly (201).

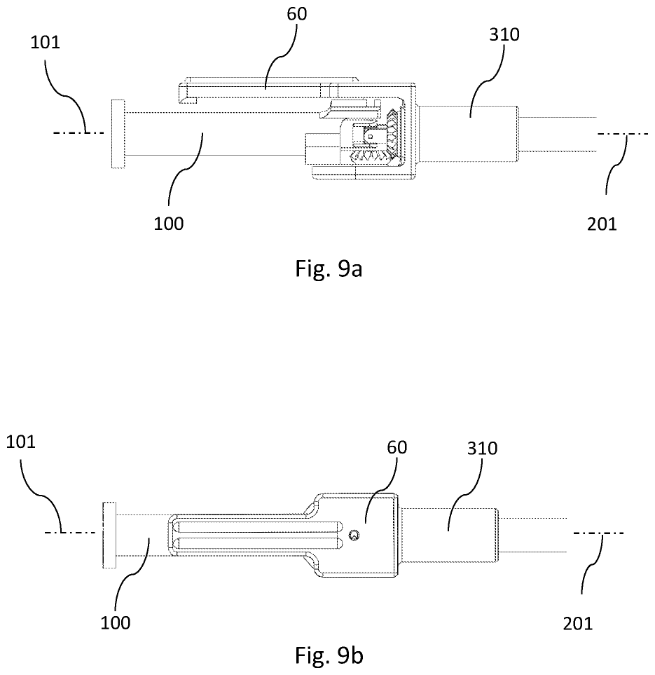

Referring to the FIGS. 9a and 9b, the first embodiment of the instrument of the present invention comprises also a brake block (60) mounted on the frame (100), said brake block (60) being configured to avoid the unintentional rotation of the frame (100) around the axis (U₃) of the connection assembly (201), when the first shaft (110), the third shaft (310) and the second shaft (210) are aligned along the axis (U₃) of the connection assembly (201).

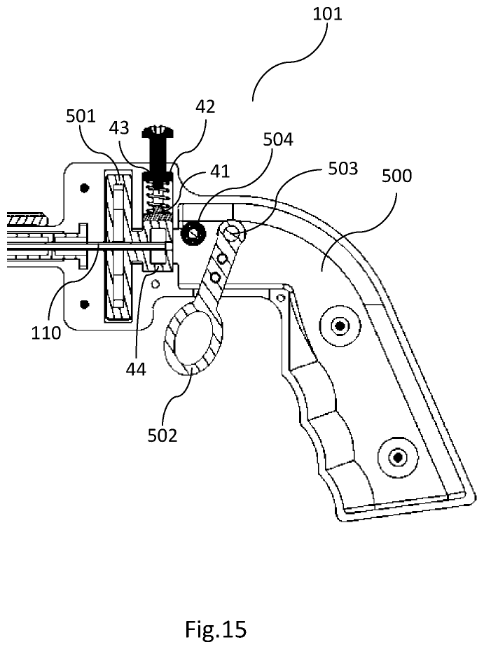

Referring to the FIGS. 2, 3, 4, 5a, 5b, 6a, 6b, 7a, 7b, 8a, 8b, 15, 19a and 19b, a second embodiment of the present invention comprises all the features mentioned above relatively to the first embodiment but, additionally, the manipulation unit (101) of the second embodiment comprises:
a handle (500, 500′);
a drum brake connecting the handle (500, 500′) to the first shaft (110, 110′, 110″), said drum brake comprising:
   a clutch pad (41);
   a compression pad (42);
   a spring (43) connecting the clutch pad and the compression pad (42); and
   a drum pulley (44) firmly connected with the first shaft (110, 110′, 110″);
a handling ring (501, 501′) firmly connected with the drum pulley (44) in such a way that a rotation of the handling ring (501, 501′) around its own axis causes a rotation of the drum pulley (44) and, thus, of the first shaft (110, 110′, 110″) around their own axes.

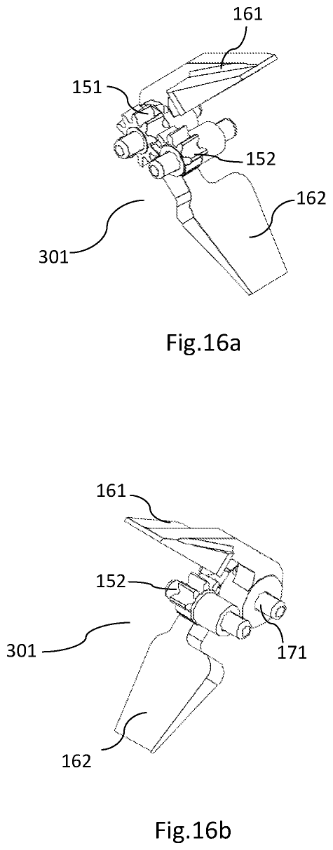

Referring to the FIGS. 2, 3, 4, 15, 16a, 16b, 19a and 19b a third embodiment of the present invention comprises:
a connection assembly (201) having an axis (U₃);
an end effector (301) mounted on a first end of the connection assembly (201), said end effector (301) comprising:
   a first arm (161);
   a second arm (162);
   a pulley (171) firmly connected with the first arm (161);
   a first gear (152) firmly connected with the second arm (162);
   a second gear (151) forming a gear assembly (151, 152) with the first gear (152), said second gear (151) being concentric and integrally connected with the pulley (171); and
a manipulation unit (101) mounted on a second end of the connection assembly (201), said manipulation unit (101) comprising:
   a trigger (502, 502′);
   a pulley (503) firmly connected with the trigger (502, 502′); and
   an idle pulley (504); and
a first shaft firmly connected with the manipulation unit (101); and
a second shaft firmly connected with the end effector (301).

In order to allow for the movement (a) of the two arms (161, 162), determining the opening and closing of the end effector (301) the instrument comprises means for transmitting motion from the manipulation unit (101) to the end effector (301), comprising a cable, not shown in the figures, which connects the pulley (503) of the manipulation unit (101) with the pulley (171) of the end effector (301), said cable being divided into two portions by means of the idle pulley (504), before to be wrapped around the pulley (503) of the manipulation unit (101).

Said cable, in particular, is inserted in a sheath of the connection assembly (201) that has a first end firmly connected to the first shaft and a second end firmly connect to second shaft.

For the purposes of the present description, the word “sheath” means “a flexible protective outside covering on a cable”. In other words, sheath and cable are similar to sheath and wire for bicycle brake transmission.

Referring to FIGS. 2, 3, 4, 5a, 5b, 6a, 6b, 7a, 7b, 8a, 8b, 15, 16a and 16b, a fourth embodiment of the present invention comprises:
a connection assembly (201) having an axis (U₃), a first end (A) and a second end (B);
an end effector (301) having an its own axis (U₂′) of rotation incident to the axis (U₃) of the connection assembly (201) at the second end (B), said end effector (301) being mounted on the second end (B) of the connection assembly (201) and comprising:
   a first arm (161);
   a second arm (162);
   a pulley (171) firmly connected with the first arm (161);
   a first gear (152) firmly connected with the second arm (162);
   a second gear (151) forming a gear assembly (151, 152) with the first gear (152), said second gear (151) being concentric and integrally connected with the pulley (171); and
a manipulation unit (101) having an its own axis (U₂) of rotation incident to the axis (U₃) of the connection assembly (201) at the first end (A), said manipulation unit (101) being mounted on the first end (A) of the connection assembly (201) and comprising:
   a trigger (502, 502′);
   a pulley (503) firmly connected with the trigger (502, 502′); and
   an idle pulley (504);
means for transmitting (111, 211, 311, 411, 310′, 110′, 210′, 410, 510, 610, 710, 411, 511, 611, 711, 811, 911) a first rotational movement of the manipulation unit (101) around a first axis (U₁) belonging to a plane orthogonal to the axis (U₃) of the connection assembly (201) and incident to the axis (U₃) of the connection assembly (201) at the first end (A) of the connection assembly (201), to the end effector (301), in such a way that the end effector (301) rotates around an axis (U₁′) orthogonal to the axis (U₃) of the connection assembly (201) and incident to the axis (U₃) of the connection assembly (201) at the second end (B) of the connection assembly (201);
means for transmitting (110, 210, 310, 400, 400′, 110″, 210″, 310″, 91, 92) a second rotational movement of the manipulation unit (101) around its own axis (U₂) to the end effector (301), in such a way that the end effector (301) rotates around its own axis (U₂′).

Referring to 2, 3, 4, 5a, 5b, 6a, 6b, 7a, 7b, 8a, 8b, 16a and 16b, the connection assembly (201) comprises a cylindrical frame (100). The means for transmitting (111, 211, 311, 411) the first rotational movement comprise:
a first bevel gear (111) firmly connected with the manipulation unit (101) and having the vertex located in correspondence with the first end (A) of the connection assembly (201);
a second bevel gear (211) forming with the first bevel gear (111) a first gear assembly, in such a way that a rotation of the first bevel gear (111) around its own axis causes a rotation of the second bevel gear (211) around its own axis;
a third bevel gear (311) firmly connected with the end effector (301) and having the vertex in correspondence with the second end (B) of the connection assembly (201);
a fourth bevel gear (411) forming with the third bevel gear (311) a second gear assembly, in such a way that a rotation of the fourth bevel gear (411) around its own axis causes a rotation of the third bevel gear (311) around its own axis; and
a hollow tube (70) aligned with the axis (U₃) of the connection assembly (201) of the connection assembly (201) and housed in the frame (100), said tube (70) cooperating with the second bevel gear (211) and the fourth bevel gear (411) in such a way that a rotation of the second bevel gear (211) around its own axis causes a rotation of the fourth bevel gear (411) around its own axis.

The means for transmitting (110, 210, 310, 400, 400′) the second rotational movement comprise:
a first shaft (110) firmly connected with the manipulation unit (101);
a second shaft (210) firmly connected with the end effector (301);
a third shaft (310) aligned with the axis (U₃) of the connection assembly (201), said third shaft (310) being housed in the tube (70);
a first Cardan joint (400) centered in correspondence of the first end (A) of the connection assembly (201); and
a second Cardan joint (400′) centered in correspondence of the second end (B) of the connection assembly (201).

In order to allow for the movement (a) of the two arms (161, 162), determining the opening and closing of the end effector (301) the instrument comprises means for transmitting motion from the manipulation unit (101) to the end effector (301), comprising a cable, not shown in the figures, which connects the pulley (503) of the manipulation unit (101) with the pulley (171) of the end effector (301), said cable being divided into two portions by means of the idle pulley (504), before to be wrapped around the pulley (503) of the manipulation unit (101). Said cable, in particular, is inserted in a sheath of the connection assembly (201) that has a first end firmly connected to the first shaft and a second end firmly connect to second shaft.

Referring to the FIGS. 9a and 9b, also the fourth embodiment of the instrument of the present invention comprises also a brake block (60) mounted on the frame (100), said brake block (60) being configured to avoid) the unintentional rotation of the frame (100) around the axis (U₃) of the connection assembly (201), when the first shaft (110), the third shaft (310) and the second shaft (210) are aligned along the axis (U₃) of the connection assembly (201).

Referring to the FIGS. 2, 3, 4, 5a, 5b, 6a, 6b, 7a, 7b, 8a, 8b, 15, 16a, 16b and 19a and 19b, a fifth embodiment of the present invention comprise all the features mentioned above relatively to the fourth embodiment but, additionally, the manipulation unit (101) of the fifth embodiment comprises:
a handle (500, 500′);
a drum brake connecting the handle (500, 500′) to the first shaft (110, 110′, 110″), said drum brake comprising:
   a clutch pad (41);
   a compression pad (42);
   a spring (43) connecting the clutch pad and the compression pad (42); and
   a drum pulley (44) firmly connected with the first shaft (110, 110′, 110″);
a handling ring (501, 501′) firmly connected with the drum pulley (44) in such a way that a rotation of the handling ring (501, 501′) around its own axis causes a rotation of the drum pulley (44) and, thus, of the first shaft (110, 110′, 110″) around their own axes.

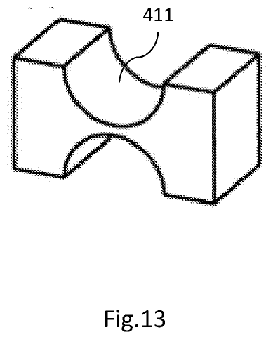

Referring to the FIGS. 2, 12, 13 and 14 in a sixth embodiment of the instrument of the present invention, the means for transmitting (310′, 110′, 210′, 410, 510, 610, 710, 411, 511, 611, 711, 811, 911) the first rotational movement comprise:
a first shaft (110′) firmly connected with the manipulation unit (101);
a second shaft (210′) firmly connected with the end effector (301);
a third shaft (310′) aligned with the axis (U₃) of the connection assembly (201), the first end of the third shaft (310′) coinciding with the first end (A) of the connection assembly (201) and the second end of the third shaft (310′) coinciding with the second end (B) of the connection assembly (201);
a fourth shaft (410) connected with the third shaft (310′) by means of a first flexure hinge (411) having the center of rotation coinciding with the first end (A) of the third shaft (310′);
a fifth shaft (510) connected with the third shaft (310′) by means of a second flexure hinge (511) having the center of rotation coinciding with the second end (B) of the third shaft (310′);
a sixth shaft (610) connected to a first portion (H₁A) of the fourth shaft (410) by means of a third flexure hinge (611) and to a first portion (H₄B) of the fifth shaft (510) by means of a fourth flexure hinge (711); and
a seventh shaft (710) connected to a second portion (AH₂) of the fourth shaft (410) by means of a fifth flexure hinge (811) and to a second portion (BH₃) of the fifth shaft (510) by means of a sixth flexure hinge (911).

The first flexure hinge (411), the second flexure hinge (511), the third flexure hinge (611), the fourth flexure hinge (711), the fifth flexure hinge (811) and the sixth flexure hinge (911) are, preferably but not exclusively, circular flexure hinges. The means for transmitting (110″, 210″, 310″, 91, 92) the second rotational movement, instead, comprise:
a first shaft (110″) firmly connected with the manipulation unit (101);
a second shaft (210″) firmly connected with the end effector (301);
a third shaft (310″) aligned with the axis (U₃) of the connection assembly (201);
a first torque coil (91) having a first end (A′) connected to a first end of the first shaft (110″) and a second end (A″) connected to a first end of the third shaft (310″), the first end (A) of the connection assembly (201) being comprised between the first end (A′) and the second end (A″) of the first torque coil (91); and
a second torque coil (92) having a first end (B′) connected to a second end of the third shaft (310″) and a second end (B″) connected to a first end of the second shaft (210″), the second end (B) of the connection assembly (201) being comprised between the first end (B′) and the second end (B″) of the second torque coil.

In order to perform the opening and closing of the end effector, the second torque coil (92) is connected to the end effector (301) by means (900) for converting a translational movement (s) of the second torque coil (92) along its own longitudinal axis, in the movement (a) of opening/closing of the two arms of the end effector, the translational movement (s) of the second torque coil (92) along its own longitudinal axis being caused by the transmission, by means of the third shaft (310″), of a translational movement of the first torque coil (91) along its own longitudinal axis. The first torque coil (91) and the second torque coil (92) can be inserted inside a first sheath (91′) and a second sheath (92′), respectively. The first shaft (110″) is housed inside a first hollow tube (800), the second shaft (210″) is housed inside a second hollow tube (802) and the third shaft (310″) is housed inside a third hollow tube (801). The first sheath (91′) has a first end that is firmly connected to the first hollow tube (800) and a second end that is firmly connected to the third hollow tube (801). The second sheath (92′) has a first end that is firmly connected to the third hollow tube (801) and a second end that is firmly connected to the second hollow tube (802).

Referring to the FIG. 2, 17, 18a, 18b, a seventh embodiment of the present invention comprises:
a connection assembly (201) having an axis (U₃), a first end (A) and a second end (B), said connection assembly comprising a frame (100);
an end effector (301) having an its own axis (U₂) of rotation incident to the axis (U₃) of the connection assembly (201) at the second end (B), said end effector (301) being mounted on the second end (B) of the connection assembly (201);
a manipulation unit (101) having an its own axis (U₂) of rotation incident to the axis (U₃) of the connection assembly (201) at the first end (A), said manipulation unit (101) being mounted on the first end (A) of the connection assembly (201);
means for transmitting at least a rotational movement of the manipulation unit (101) around its own axis (U₂), to the end effector (301), in such a way that the end effector (301) rotates around its own axis (U₂′), the axis (U₂′), of the end effector (301) being parallel to the axis (U₂) of the manipulation unit (101);
a blocking device (50) mounted on the frame (100) configured to avoid the unintentional rotation of the frame (100) around the axis (U₃) of the connection assembly (201), said device (50) comprising:
   a support structure (30) with means for docking (31, 32) the device (50) to an instrument holder (80) with which the instrument can cooperate, said instrument holder (80) being configured to be at least partial inserted of said laparoscopic surgical instrument through an incision into a patient cavity;
   two truncated cone rolls (81, 82), the frame (100″) being placed between the two truncated cone rolls (81, 82); and
   a lever (70) configured to allow the two truncated cone rolls (81, 82) to translate from a first position in which the two truncated cone rolls (81, 82) are not in contact with the frame (100) to a second position in which the two truncated cone rolls (81, 82) are in contact with the frame (100) preventing by friction the rotation of the frame (100) around the axis (U₃) of the connection assembly (201), the translation of the frame (100) along the axis (U₃) of the connection assembly (201) being maintained allowable.

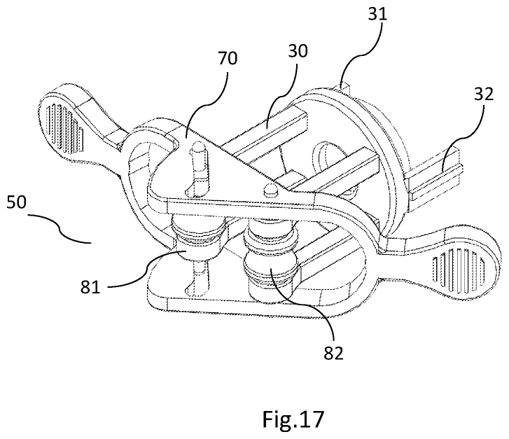

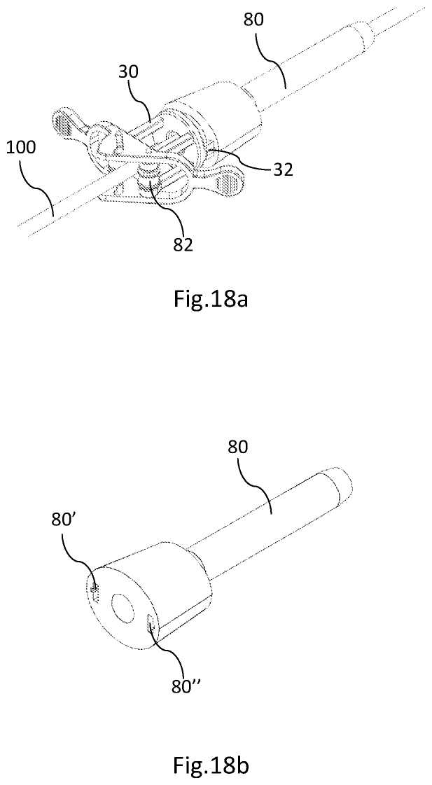

Referring to the FIGS. 2, 2, 3, 4, 5a, 5b, 6a, 6b, 7a, 7b, 8a, 8b, 17, 18a and 18b, an eight embodiment of the instrument of the present invention comprises:
a connection assembly (201) having an axis (U₃), said connection assembly (201) comprising a frame (100);
an end effector (301) having an its own axis (U₂′) of rotation incident to the axis (U₃) of the connection assembly (201) at the second end (B), said end effector (301) being mounted on the second end (B) of the connection assembly (201);
a manipulation unit (101) having an its own axis (U₂) of rotation incident to the axis (U₃) of the connection assembly (201) at the first end (A), said manipulation unit (101) being mounted on the first end (A) of the connection assembly (201);
a blocking device (50) mounted on the frame (100) configured to avoid the unintentional rotation of the frame (100) around the axis (U₃) of the connection assembly (201), said device (50) comprising:
   a support structure (30) with means for docking (31, 32) the device (50) to an instrument holder (80) with which the instrument can cooperate, said instrument holder (80) being configured to be at least partial inserted of said laparoscopic surgical instrument through an incision into a patient cavity;
   two truncated cone rolls (81, 82), the frame (100″) being placed between the two truncated cone rolls (81, 82); and
   a lever (70) configured to allow the two truncated cone rolls (81, 82) to translate from a first position in which the two truncated cone rolls (81, 82) are not in contact with the frame (100) to a second position in which the two truncated cone rolls (81, 82) are in contact with the frame (100) preventing by friction the rotation of the frame (100) around the axis (U₃) of the connection assembly (201), the translation of the frame (100) along the axis (U₃) of the connection assembly (201) being maintained allowable;
means for transmitting (111, 211, 311, 411, 310′, 110′, 210′, 410, 510, 610, 710, 411, 511, 611, 711, 811, 911) a first rotational movement of the manipulation unit (101) around a first axis (U₁) belonging to a plane orthogonal to the axis (U₃) of the connection assembly (201) and incident to the axis (U₃) of the connection assembly (201) at the first end (A) of the connection assembly (201), to the end effector (301), in such a way that the end effector (301) rotates around an axis (U₁′) orthogonal to the axis (U₃) of the connection assembly (201) and incident to the axis (U₃) of the connection assembly (201) at the second end (B) of the connection assembly (201); and
means for transmitting (110, 210, 310, 400, 400′, 110″, 210″, 310″, 91, 92) a second rotational movement of the manipulation unit (101) around its own axis (U₂) to the end effector (301), in such a way that the end effector (301) rotates around its own axis (U₂′), the axis (U₂′), of the end effector (301) being parallel to the axis (U₂) of the manipulation unit (101).

The connection assembly (201) comprises a cylindrical frame (100). The means for transmitting (111, 211, 311, 411) the first rotational movement comprise:
a first bevel gear (111) firmly connected with the manipulation unit (101) and having the vertex located in correspondence with the first end (A) of the connection assembly (201);
a second bevel gear (211) forming with the first bevel gear (111) a first gear assembly, in such a way that a rotation of the first bevel gear (111) around its own axis causes a rotation of the second bevel gear (211) around its own axis;
a third bevel gear (311) firmly connected with the end effector (301) and having the vertex in correspondence with the second end (B) of the connection assembly (201);
a fourth bevel gear (411) forming with the third bevel gear (311) a second gear assembly, in such a way that a rotation of the fourth bevel gear (411) around its own axis causes a rotation of the third bevel gear (311) around its own axis; and
a hollow tube (70) aligned with the axis (U₃) of the connection assembly (201) and housed in the frame (100), said tube (70) cooperating with the second bevel gear (211) and the fourth bevel gear (411) in such a way that a rotation of the second bevel gear (211) around its own axis causes a rotation of the fourth bevel gear (411) around its own axis.

The means for transmitting (110, 210, 310, 400, 400′) the second rotational movement comprise:
a first shaft (110) firmly connected with the manipulation unit (101);
a second shaft (210) firmly connected with the end effector (301);
a third shaft (310) aligned with the axis (U₃) of the connection assembly (201), said third shaft (310) being housed in the tube (70);
a first Cardan joint (400) centered in correspondence of the first end (A) of the connection assembly (201); and
a second Cardan joint (400′) centered in correspondence of the second end (B) of the connection assembly (201).

Referring to the FIGS. 9a and 9b, also the eight embodiment of the instrument of the present invention comprises also a brake block (60) mounted on the frame (100), said brake block (60) being configured to avoid the unintentional rotation of the frame (100) around the axis (U₃) of the connection assembly (201), when the first shaft (110), the third shaft (310) and the second shaft (210) are aligned along the axis (U₃) of the connection assembly.

Referring to the FIGS. 2, 3, 4, 5a, 5b, 6a, 6b, 7a, 7b, 8a, 8b, 15, 17, 18a, 18b, 19a and 19b, a ninth embodiment of the present invention comprises all the features mentioned above relatively to the eight embodiment but, additionally, the manipulation unit (101) of the ninth embodiment comprises:
a handle (500, 500′);
a drum brake connecting the handle (500, 500′) to the first shaft (110, 110′, 110″), said drum brake comprising:
   a clutch pad (41);
   a compression pad (42);
   a spring (43) connecting the clutch pad and the compression pad (42); and
   a drum pulley (44) firmly connected with the first shaft (110, 110′, 110″);
a handling ring (501, 501′) firmly connected with the drum pulley (44) in such a way that a rotation of the handling ring (501, 501′) around its own axis causes a rotation of the drum pulley (44) and, thus, of the first shaft (110, 110′, 110″) around their own axes.

Referring to the FIGS. 2, 3, 4, 15, 16a, 16b, 17, 18a, and 18b, a tenth embodiment of the present invention comprises:
a connection assembly (201) having an axis (U₃) of the connection assembly (201) and comprising a frame (100);
an end effector (301) having an its own axis (U₂) of rotation incident to the axis (U₃) of the connection assembly (201) at the second end (B), said end effector (301) being mounted on the second end (B) of the connection assembly (201) and comprising:
   a first arm (161);
   a second arm (162);
   a pulley (171) firmly connected with the first arm (161);
   a first gear (152) firmly connected with the second arm (162);
   a second gear (151) forming a gear assembly (151, 152) with the first gear (152), said second gear (151) being concentric and integrally connected with the pulley (171); and
a manipulation unit (101) having an its own axis (U₂) of rotation incident to the axis (U₃) of the connection assembly (201) at the first end (A), said manipulation unit (101) being mounted on the first end (A) of the connection assembly (201) and comprising:
   a trigger (502, 502′);
   a pulley (503) firmly connected with the trigger (502, 502′); and
   an idle pulley (504);
a first shaft firmly connected with the manipulation unit (101);
a second shaft firmly connected with the end effector (301);
means for transmitting at least a rotational movement of the manipulation unit (101) around its own axis (U₂), to the end effector (301), in such a way that the end effector (301) rotates around its own axis (U₂′), the axis (U₂′), of the end effector (301) being parallel to the axis (U₂) of the manipulation unit (101);
a blocking device (50) mounted on the frame (100) configured to avoid the unintentional rotation of the frame (100) around the axis (U₃) of the connection assembly (201), said device (50) comprising:
   a support structure (30) with means for docking (31, 32) the device (50) to the instrument holder (80);
   two truncated cone rolls (81, 82), the frame (100″) being placed between the two truncated cone rolls (81, 82); and
   a lever (70) configured to allow the two truncated cone rolls (81, 82) to translate from a first position in which the two truncated cone rolls (81, 82) are not in contact with the frame (100) to a second position in which the two truncated cone rolls (81, 82) are in contact with the frame (100) preventing by friction the rotation of the frame (100) around the axis (U₃) of the connection assembly (201), the translation of the frame (100) along the axis (U₃) of the connection assembly (201) being maintained allowable.

In order to allow for the movement (a) of the two arms (161, 162), determining the opening and closing of the end effector (301) the instrument comprises means for transmitting motion from the manipulation unit (101) to the end effector (301), comprising a cable, not shown in the figures, which connects the pulley (503) of the manipulation unit (101) with the pulley (171) of the end effector (301). Said cable, in particular, is inserted in a sheath of the connection assembly (201) that has a first end firmly connected to the first shaft and a second end firmly connect to second shaft. Said cable comprises two branches that wrap around the pulley (503) of the manipulation unit (101) and the pulley (171) of the end effector (301), the two branches being separated by means of the idle pulley (504), before to be wrapped around the pulley (503) of the manipulation unit (101). Before to be wrapped around the pulley (171) of the end effector, instead, the two branches are separated because they slide on the walls of the sheath itself.

Referring to FIGS. 2, 3, 4, 5a, 5b, 6a, 6b, 7a, 7b, 8a, 8b, 15, 16a, 16b, 17, 18a, and 18b, an eleventh embodiment of the present invention comprises:
a connection assembly (201) having an axis (U₃), a first end (A) and a second end (B);
an end effector (301) having an its own axis (U₂′) of rotation incident to the axis (U₃) of the connection assembly (201) at the second end (B), said end effector (301) being mounted on the second end (B) of the connection assembly (201) and comprising:
   a first arm (161);
   a second arm (162);
   a pulley (171) firmly connected with the first arm (161);
   a first gear (152) firmly connected with the second arm (162);
   a second gear (151) forming a gear assembly (151, 152) with the first gear (152), said second gear (151) being concentric and integrally connected with the pulley (171); and
a manipulation unit (101) having an its own axis (U₂) of rotation incident to the axis (U₃) of the connection assembly (201) at the first end (A), said manipulation unit (101) being mounted on the first end (A) of the connection assembly (201) and comprising:
   a trigger (502, 502′);
   a pulley (503) firmly connected with the trigger (502, 502′); and
   an idle pulley (504);
means for transmitting (111, 211, 311, 411, 310′, 110′, 210′, 410, 510, 610, 710, 411, 511, 611, 711, 811, 911) a first rotational movement of the manipulation unit (101) around a first axis (U₁) belonging to a plane orthogonal to the axis (U₃) of the connection assembly (201) and incident to the axis (U₃) of the connection assembly (201) at the first end (A) of the connection assembly (201), to the end effector (301), in such a way that the end effector (301) rotates around an axis (U₁′) orthogonal to the axis (U₃) of the connection assembly (201) and incident to the axis (U₃) of the connection assembly (201) at the second end (B) of the connection assembly (201);
means for transmitting (110, 210, 310, 400, 400′, 110″, 210″, 310″, 91, 92) a second rotational movement of the manipulation unit (101) around it own axis (U₂) to the end effector (301), in such a way that the end effector (301) rotates around it own axis (U₂′); and
a blocking device (50) mounted on the frame (100) configured to avoid the unintentional rotation of the frame (100) around the axis (U₃) of the connection assembly (201), said device (50) comprising:
   a support structure (30) with means for docking (31, 32) the device (50) to the instrument holder (80);
   two truncated cone rolls (81, 82), the frame (100″) being placed between the two truncated cone rolls (81, 82); and
   a lever (70) configured to allow the two truncated cone rolls (81, 82) to translate from a first position in which the two truncated cone rolls (81, 82) are not in contact with the frame (100) to a second position in which the two truncated cone rolls (81, 82) are in contact with the frame (100) preventing by friction the rotation of the frame (100) around the axis (U₃) of the connection assembly (201), the translation of the frame (100) along the axis (U₃) of the connection assembly (201) being maintained allowable.

Referring to 2, 3, 4, 5a, 5b, 6a, 6b, 7a, 7b, 8a, 8b, 16a and 16b, the connection assembly (201) comprises a cylindrical frame (100). The means for transmitting (111, 211, 311, 411) the first rotational movement comprise:
a first bevel gear (111) firmly connected with the manipulation unit (101) and having the vertex located in correspondence with the first end (A) of the connection assembly (201);
a second bevel gear (211) forming with the first bevel gear (111) a first gear assembly, in such a way that a rotation of the first bevel gear (111) around its own axis causes a rotation of the second bevel gear (211) around its own axis;
a third bevel gear (311) firmly connected with the end effector (301) and having the vertex in correspondence with the second end (B) of the connection assembly (201);
a fourth bevel gear (411) forming with the third bevel gear (311) a second gear assembly, in such a way that a rotation of the fourth bevel gear (411) around its own axis causes a rotation of the third bevel gear (311) around its own axis; and
a hollow tube (70) aligned with the axis (U₃) of the connection assembly (201) and housed in the frame (100), said tube (70) cooperating with the second bevel gear (211) and the fourth bevel gear (411) in such a way that a rotation of the second bevel gear (211) around its own axis causes a rotation of the fourth bevel gear (411) around its own axis.

The means for transmitting (110, 210, 310, 400, 400′) the second rotational movement comprise:
a first shaft (110) firmly connected with the manipulation unit (101);
a second shaft (210) firmly connected with the end effector (301);
a third shaft (310) aligned with the axis (U₃) of the connection assembly (201), said third shaft (310) being housed in the tube (70);
a first Cardan joint (400) centered in correspondence of the first end (A) of the connection assembly (201); and
a second Cardan joint (400′) centered in correspondence of the second end (B) of the connection assembly (201).

In order to allow for the movement (a) of the two arms (161, 162), determining the opening and closing of the end effector (301) the instrument comprises means for transmitting motion from the manipulation unit (101) to the end effector (301), comprising a cable, not shown in the figures, which connects the pulley (503) of the manipulation unit (101) with the pulley (171) of the end effector (301). Said cable, in particular, is inserted in a sheath of the connection assembly (201) that has a first end firmly connected to the first shaft and a second end firmly connect to second shaft. Said cable comprises two branches that wrap around the pulley (503) of the manipulation unit (101) and the pulley (171) of the end effector (301), the two branches being separated by means of the idle pulley (504) before to be wrapped around the pulley (503) of the manipulation unit (101). Before to be wrapped around the pulley (171) of the end effector, instead, the two branches are separated because they slide on the walls of the sheath itself.

Referring to the FIGS. 9a and 9b, also the eleventh embodiment of the instrument of the present invention comprises also a brake block (60) mounted on the frame (100), said brake block (60) being configured to avoid the unintentional rotation of the frame (100) around the axis (U₃) of the connection assembly (201), when the first shaft (110), the third shaft (310) and the second shaft (210) are aligned along the axis (U₃) of the connection assembly (201).

Referring to the FIGS. 2, 3, 4, 5a, 5b, 6a, 6b, 7a, 7b, 8a, 8b, 15, 16a, 16b and 19a and 19b, a twelfth embodiment of the present invention comprise all the features mentioned above relatively to the eleventh embodiment but, additionally, the manipulation unit (101) of the twelfth embodiment comprises:
a handle (500, 500′);
a drum brake connecting the handle (500, 500′) to the first shaft (110, 110′, 110″), said drum brake comprising:
   a clutch pad (41);
   a compression pad (42);
   a spring (43) connecting the clutch pad and the compression pad (42); and
   a drum pulley (44) firmly connected with the first shaft (110, 110′, 110″);
a handling ring (501, 501′) firmly connected with the drum pulley (44) in such a way that a rotation of the handling ring (501, 501′) around its own axis causes a rotation of the drum pulley (44) and, thus, of the first shaft (110, 110′, 110″) around their own axes.

Referring to the FIGS. 2, 12, 13 and 14, 17, 18a, and 18b, in a thirteenth embodiment of the instrument of the present invention comprises:
a connection assembly (201) having an axis (U₃), said connection assembly (201) comprising a frame (100);
an end effector (301) having an its own axis (U₂′) of rotation incident to the axis (U₃) of the connection assembly (201) at the second end (B), said end effector (301) being mounted on the second end (B) of the connection assembly (201);
a manipulation unit (101) having an its own axis (U₂) of rotation incident to the axis (U₃) of the connection assembly (201) at the first end (A), said manipulation unit (101) being mounted on the first end (A of the connection assembly (201);
a blocking device (50) mounted on the frame (100) configured to avoid the unintentional rotation of the frame (100) around the axis (U₃) of the connection assembly (201), said device (50) comprising:
   a support structure (30) with means for docking (31, 32) the device (50) to an instrument holder (80) with which the instrument can cooperate, said instrument holder (80) being configured to be at least partial inserted of said laparoscopic surgical instrument through an incision into a patient cavity;
   two truncated cone rolls (81, 82), the frame (100″) being placed between the two truncated cone rolls (81, 82); and
   a lever (70) configured to allow the two truncated cone rolls (81, 82) to translate from a first position in which the two truncated cone rolls (81, 82) are not in contact with the frame (100) to a second position in which the two truncated cone rolls (81, 82) are in contact with the frame (100) preventing by friction the rotation of the frame (100) around the axis (U₃) of the connection assembly (201), the translation of the frame (100) along the axis (U₃) of the connection assembly (201) being maintained allowable;
means for transmitting (111, 211, 311, 411, 310′, 110′, 210′, 410, 510, 610, 710, 411, 511, 611, 711, 811, 911) a first rotational movement of the manipulation unit (101) around a first axis (U₁) belonging to a plane orthogonal to the axis (U₃) of the connection assembly (201) and incident to the axis (U₃) of the connection assembly (201) at the first end (A) of the connection assembly (201), to the end effector (301), in such a way that the end effector (301) rotates around an axis (U₁′) orthogonal to the axis (U₃) of the connection assembly (201) and incident to the axis (U₃) of the connection assembly (201) at the second end (B) of the connection assembly (201); and
means for transmitting (110, 210, 310, 400, 400′, 110″, 210″, 310″, 91, 92) a second rotational movement of the manipulation unit (101) around its own axis (U₂) to the end effector (301), in such a way that the end effector (301) rotates around its own axis (U_{2′}), the axis (U_{2′}), of the end effector (301) being parallel to the axis (U₂) of the manipulation unit (101).

The means for transmitting (310′, 110′, 210′, 410, 510, 610, 710, 411, 511, 611, 711, 811, 911) the first rotational movement comprise:
a first shaft (110′) firmly connected with the manipulation unit (101);
a second shaft (210′) firmly connected with the end effector (301);
a third shaft (310′) aligned with the axis (U₃) of the connection assembly (201), the first end of the third shaft (310′) coinciding with the first end (A) of the connection assembly (201) and the second end of the third shaft (310′) coinciding with the second end (B) of the connection assembly (201);
a fourth shaft (410) integrally connected with the first shaft (110′), said fourth shaft (510) being connected with the third shaft (310′) by means of a first flexure hinge (411) having the center of rotation coinciding with the first end (A) of the third shaft (310′);
a fifth shaft (510) integrally connected with the second shaft (210′), said fifth shaft (510) being connected with the third shaft (310′) by means of a second flexure hinge (511) having the center of rotation coinciding with the second end (B) of the third shaft (310′);
a sixth shaft (610) connected to a first portion (H₁A) of the fourth shaft (410) by means of a third flexure hinge (611) and to a first portion (H₄B) of the fifth shaft (510) by means of a fourth flexure hinge (711); and
a seventh shaft (710) connected to a second portion (AH₂) of the fourth shaft (410) by means of a fifth flexure hinge (811) and to a second portion (BH₃) of the fifth shaft (510) by means of a sixth flexure hinge (911).

The first flexure hinge (411), the second flexure hinge (511), the third flexure hinge (611), the fourth flexure hinge (711), the fifth flexure hinge (811) and the sixth flexure hinge (911) are, preferably but not exclusively, circular flexure hinges. The means for transmitting (110″, 210″, 310″, 91, 92) the second rotational movement, instead, comprise:
a first shaft (110″) firmly connected with the manipulation unit (101);
a second shaft (210″) firmly connected with the end effector (301);
a third shaft (310″) aligned with the axis (U₃) of the connection assembly (201);
a first torque coil (91) having a first end (A′) connected to a first end of the first shaft (110″) and a second end (A″) connected to a first end of the third shaft (310″), the first end (A) of the connection assembly (201) being comprised between the first end (A′) and the second end (A″) of the first torque coil (91); and
a second torque coil (92) having a first end (B′) connected to a second end of the third shaft (310″) and a second end (B″) connected to a first end of the second shaft (210″), the second end (B) of the connection assembly (201) being comprised between the first end (B′) and the second end (B″) of the second torque coil.

In order to perform the opening and closing of the end effector the instrument comprises second torque coil (92) connected to the end effector (301) by means (900) for converting a translational movement (s) of the second torque coil (92) along its own longitudinal axis, in the movement (a) of opening/closing of the two arms of the end effector, the translational movement (s) of the second torque coil (92) along its own longitudinal axis being caused by the transmission, by means of the third shaft (310″), of a translational movement of the first torque coil (91) along its own longitudinal axis. The first torque coil (91) and the second torque coil (92) can be inserted inside a first sheath (91′) and second sheath (92′), respectively. The first shaft (110″) is housed inside a first hollow tube (800), the second shaft (210″) is housed inside a second hollow tube (802) and the third shaft (310″) is housed inside a third hollow tube (801). The first sheath (91′) has a first end that is firmly connected to the first hollow tube (800) and a second end that is firmly connected to the third hollow tube (801). The second sheath (92′) has a first end that is firmly connected to the third hollow tube (801) and a second end that is firmly connected to the second hollow tube (802).

In all the embodiments of the instrument detailed above, the end effector (301) can be a clamp (301′, 301′) or a scissor or a grasper.

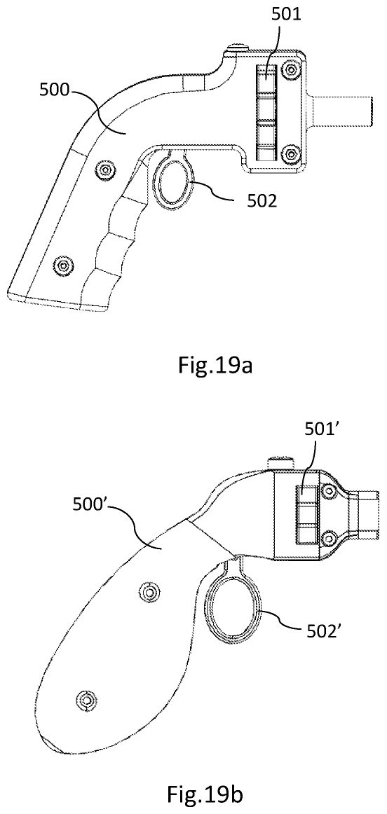

Referring to FIGS. 19a and 19b, the manipulation unit (101), can be designed according two different embodiments. The first one (500) is showed in FIG. 19a and the second one (500′) is showed in FIG. 19b. The second embodiment of the handle (500′), differently from the first one (500), has an ergonomic shape that allows the surgeons to move the ring (501′) with the thumb of the same hand used for maneuvering the handle (500) itself.

Finally, here it is specified that it forms subject matter of the present invention also a kit for laparoscopy interventions comprising:
a laparoscopic surgical instrument according to any of the embodiment described above from the seventh to thirteenth one;
   an instrument holder (80) for at least a partial insertion of said laparoscopic surgical instrument through an incision into a patient cavity, said instrument holder (80) being provided with means for docking (80′, 80″) the instrument holder (80) to the blocking device (50), the means for docking (80′, 80″) the instrument holder (80) being configured to cooperate with the means for docking (31, 32) of the support structure (30) of the blocking device (50).

1. A laparoscopic surgical instrument, comprising:
a connection assembly having an axis, a first end, and a second end;
an end effector having an axis of rotation incident to the axis of the connection assembly at the second end of the connection assembly, said end effector being mounted on the second end of the connection assembly;
a manipulation unit having an axis of rotation incident to the axis of the connection assembly at the first end of the connection assembly, said manipulation unit being mounted on the first end of the connection assembly; and
a means for transmitting motion from the manipulation unit to the end effector;
wherein the means for transmitting motion comprises at least one of:
a means for transmitting, to the end effector, a first rotational movement of the manipulation unit around a first axis that is along a plane orthogonal to the axis of the connection assembly and incident to the axis of the connection assembly at the first end of the connection assembly, in such a way that the end effector rotates around an axis orthogonal to the axis of the connection assembly and incident to the axis of the connection assembly at the second end of the connection assembly; and
a means for transmitting, to the end effector, a second rotational movement of the manipulation unit around its axis, in such a way that the end effector rotates around its axis parallel to the axis of the manipulation unit.

2. The instrument according to claim 1, wherein the means for transmitting the first rotational movement comprises:
a first bevel gear firmly connected with the manipulation unit and having the vertex located in correspondence with the first end of the connection assembly;
a second bevel gear forming with the first bevel gear a first gear assembly, in such a way that a rotation of the first bevel gear around an axis of the first bevel gear causes a rotation of the second bevel gear around an axis of the second bevel gear;
a third bevel gear firmly connected with the end effector and having the vertex in correspondence with the second end of the connection assembly;
a fourth bevel gear forming with the third bevel gear a second gear assembly, in such a way that a rotation of the fourth bevel gear around an axis of the fourth bevel gear causes a rotation of the third bevel gear around an axis of the third bevel gear; and
a tube aligned with the axis of the connection assembly, said tube cooperating with the second bevel gear and the fourth bevel gear in such a way that a rotation of the second bevel gear around the axis of the second bevel gear causes a rotation of the fourth bevel gear around the axis of the fourth bevel gear.

3. The instrument according to claim 2, wherein the means for transmitting the second rotational movement comprises:
a first shaft firmly connected with the manipulation unit;
a second shaft firmly connected with the end effector;
a third shaft aligned with the axis of the connection assembly;
a first Cardan joint centered in correspondence of the first end of the connection assembly; and
a second Cardan joint centered in correspondence of the second end of the connection assembly.

4. The instrument according to claim 3, wherein the connection assembly comprises a frame housing the tube, and wherein the tube is a hollow tube that houses the third shaft.

5. The instrument according to claim 4, comprising a brake block mounted on the frame, said brake block being configured to avoid the unintentional rotation of the frame around the axis of the connection assembly, when the first shaft, the third shaft and the second shaft are aligned along the axis of the connection assembly.

6. The instrument according to claim 1, wherein the means for transmitting the first rotational movement comprises:
a first shaft firmly connected with the manipulation unit;
a second shaft firmly connected with the end effector;
a third shaft aligned with the axis of the connection assembly, wherein a first end of the third shaft coincides with the first end of the connection assembly, and wherein a second end of the third shaft coincides with the second end of the connection assembly;
a fourth shaft integrally connected with the first shaft, said fourth shaft being connected with the third shaft by a first flexure hinge having a center of rotation coinciding with the first end of the third shaft;
a fifth shaft integrally connected with the second shaft, said fifth shaft being connected with the third shaft by a second flexure hinge having a center of rotation coinciding with the second end of the third shaft;
a sixth shaft connected to a first portion of the fourth shaft by a third flexure hinge, wherein the sixth shaft is connected to a first portion of the fifth shaft by a fourth flexure hinge; and
a seventh shaft connected to a second portion of the fourth shaft by a fifth flexure hinge, wherein the seventh shaft is connected to a second portion of the fifth shaft by a sixth flexure hinge.

7. The instrument according to the claim 6, wherein the first flexure hinge, the second flexure hinge, the third flexure hinge, the fourth flexure hinge, the fifth flexure hinge and the sixth flexure hinge comprise a circular flexure hinges.

8. The instrument according to claim 1, wherein the means for transmitting the second rotational movement comprises:
a first shaft connected to the manipulation unit;
a second shaft connected to the end effector;
a third shaft aligned with the axis of the connection assembly;
a first torque coil having a first end and a second end, wherein the first end of the first torque coil is connected to a first end of the first shaft, wherein the second end of the first torque coil is connected to a first end of the third shaft, and wherein the first end of the connection assembly is positioned between the first end of the first torque coil and the second end of the first torque coil; and
a second torque coil having a first end and a second end, wherein the first end of the second torque coil is connected to a second end of the third shaft, wherein the second end of the second torque coil is connected to a first end of the second shaft, wherein the second end of the connection assembly is positioned between the first end of the second torque coil and the second end of the second torque coil.

9. The instrument according to claim 8, wherein the second torque coil is connected to the end effector and configured to convert a translational movement of the second torque coil along a longitudinal axis of the second torque coil to open and/or close the two arms of the end effector, wherein the translational movement of the second torque coil is caused by transmission of the third shaft, of a translational movement of the first torque coil along a longitudinal axis of the first torque coil.

10. The instrument according to claim 3, wherein the manipulation unit comprises:
a handle;
a drum brake connecting the handle to the first shaft, said drum brake comprising:
a clutch pad;
a compression pad;
a spring connecting the clutch pad and the compression pad; and
a drum pulley firmly connected with the first shaft;
a handling ring firmly connected with the drum pulley in such a way that a rotation of the handling ring around an axis of the handling ring causes a rotation of the drum pulley around an axis of the drum pulley and causes a rotation of the first shaft around an axis of the first shaft.

11. A laparoscopic surgical instrument, comprising:
a connection assembly having an axis;
an end effector mounted on a first end of the connection assembly, said end effector comprising a first arm and a second arm;
a manipulation unit mounted on a second end of the connection assembly, said manipulation unit comprising a trigger, a pulley firmly connected with the trigger, and an idle pulley;
a means for transmitting motion from the manipulation unit to the end effector;
a first shaft firmly connected to the manipulation unit; and
a second shaft connected to the end effector;
wherein the end effector comprises:
a first arm;
a second arm;
a pulley connected to the first arm;
a first gear connected to the second arm; and
a second gear forming a gear assembly with the first gear, wherein the second gear is concentric and integrally connected with the pulley;
wherein the means for transmitting motion comprises a cable connecting the pulley of the manipulation unit with the pulley of the end effector, said cable comprising two branches that wrap around each of the pulley of the manipulation unit and the pulley of the end effector, wherein the two branches are separated by the idle pulley.

12. The instrument according to claim 11, wherein the connection assembly further comprises a sheath having a first end connected to the first shaft and a second end connected to the second shaft, wherein the two branches of the cable are separated from one another.

13. The instrument according to claim 1, wherein the end effector is at least one of a clamp, or a scissor, and a grasper.

14. A laparoscopic surgical instrument suitable to cooperate with an instrument holder for at least a partial insertion of said laparoscopic surgical instrument through an incision into a patient cavity, said instrument comprising:
a connection assembly having an axis, a first end, and a second end, wherein the connection assembly comprises a frame;
an end effector having an axis of rotation incident to the axis of the connection assembly at the second end of the connection assembly, wherein the end effector is mounted on the second end of the connection assembly;
a manipulation unit having an axis of rotation incident to the axis of the connection assembly at the first end of the connection assembly, wherein the manipulation unit is mounted on the first end of the connection assembly;
a means for transmitting at least a rotational movement of the manipulation unit around the axis of the manipulation unit, to the end effector, in such a way that the end effector rotates around the axis of the end effector, wherein the axis of the end effector is parallel to the axis of the manipulation unit; and
a blocking device mounted on the frame and configured to avoid the unintentional rotation of the frame around the axis of the connection assembly, wherein the blocking device comprises:
a support structure with means for docking the blocking device to the instrument holder;
two truncated cone rolls, wherein the frame is placed between the two truncated cone rolls; and
a lever configured to allow the two truncated cone rolls to translate from a first position in which the two truncated cone rolls are not in contact with the frame to a second position in which the two truncated cone rolls are in contact with the frame preventing, by friction, the rotation of the frame around the axis of the connection assembly, such that the translation of the frame along the axis of the connection assembly is maintained as allowable.

15. Kit for laparoscopy interventions comprising:
a laparoscopic surgical instrument; and
an instrument holder for at least a partial insertion of said laparoscopic surgical instrument through an incision into a patient cavity, wherein the instrument holder has a means for docking the instrument holder to a blocking device that has a support structure, wherein the support structure has means for docking the blocking device to the instrument holder, and wherein the means for docking the instrument holder to the blocking device is configured to cooperate with the means of the support structure for docking the blocking device to the instrument holder.
